(19) United States
(12) Patent Application Publication
(10) Pub. No.: US 2003/0104750 A1
(43) Pub. Date: Jun. 5, 2003

(54) Personal care and surface cleaning article

(76) Inventor: Albert R. Kelly, New York, NY (US)

Correspondence Address: Evelyn M. Sommer, 30th Floor, 825 Third Avenue, New York, NY, 10022, US

(21) Appl. No.: 10/021,395

(22) Filed: Dec. 19, 2001

(51) Int. Cl. B32B 5/26; D04H 13/00; D04H 5/00; D04H 3/00; D04H 1/00

(52) U.S. Cl. 442/381; 442/327

(57) ABSTRACT

Flexible, substantially dry, disposable article suitable as a personal care article for cleaning and conditioning skin, and for cleaning, sanitizing and disinfecting hard surfaces, methods of manufacturing and using the same wherein the article comprises: a first water insoluble flexible nonwoven thermoplastic outer layer having an outer and inner surface; a core layer containing a treatment composition adapted for cleaning, sanitizing or disinfecting hard surfaces or for personal cleaning and conditioning and a second water insoluble flexible nonwoven thermoplastic layer having an outer and inner surface positioned so its inner surface is opposite the inner surface of the first layer, the core layer having been ultrasonically bonded to and between the first and second layers so as to form a unified article having pin-dot perforations of less than 0.5 millimeters in diameter formed ultrasonically extending through all three of said layers.

The present invention relates to a substantially dry, flexible disposable article suitable as a personal care article for cleaning and conditioning skin and for cleaning, sanitizing and disinfecting hard surfaces, methods for manufacturing and using the same.

Treated flexible articles for personal care and for cleaning, sanitizing and disinfecting hard surfaces are known. These articles are generally single ply structures that have been coated, sprayed or impregnated with a treatment composition that is activated on being wetted with water. The articles with the treatment composition incorporated therein are substantially dry to the touch. A disadvantage of these articles is when large surfaces are involved or when it is desired that the article be used for more than one use, necessitating the rewetting of the article to reactivate the treatment composition incorporated therein, that on the first wetting of the article, the active ingredients are substantially completely released such that the intended larger or multi stage cleaning task cannot be completed. Another disadvantage of the available cleaning articles is that the use of higher levels of treatment composition adversely affects hand-feel, and the heavily treated articles feel wet, lubricious or boardy.

A key requisite for producing longer-lasting cleaning articles is inclusion of enough treatment composition for the tasks intended, and provisions both for accelerating activation of that composition by water and for controlling the release of the activated treatment composition over time. Traditionally, this had been accomplished with the use of concentrated treatment compositions that contain little or no water, that are solid or semi-solid at room temperatures, that are coated onto the surface of a flexible substrate at elevated temperatures, and that are allowed to dry as a thin coating at ambient temperatures. The disadvantages of these types of coatings include lubricity of the wax-like coatings, boardy hand-feel and an inability to control activation or release; all of which depend upon the chemistry of the treatment compositions.

An object of this invention is to provide a substantially dry, flexible cleaning article that can be activated with water and reactivated for continued or repeated use until the cleaning task is or tasks are completed.

Another objective of this invention is a flexible, substantially dry, cleaning article having incorporated into its structure treatment composition in an amount of from 25 to 300 percent of the structure's total basis weight without the hand feel being adversely affected.

It is another object of the invention to provide such cleaning articles which are disposable.

Still another object is a cleaning article adapted for controlled and repeated release of the active ingredients present in the treatment composition incorporated in its structure.

Yet another object is to provide a cleaning article adapted for controlled and repeated release of the active ingredients present in the treatment composition incorporated in its structure adapted for use for personal care.

Yet another object is to provide a cleaning article adapted for controlled and repeated release of the active ingredients present in the treatment composition incorporated in its structure adapted for use for cleaning, sanitizing and disinfecting hard surfaces.

Still a further object of the invention is to provide a cleaning article adapted for controlled and repeated release of the active ingredients present in the treatment composition incorporated in its structure having different surface textures on opposite sides thereof.

Yet another object of the invention is to provide a cleaning article adapted for controlled and repeated release of the active ingredients in the treatment composition incorporated in its structure having an outer surface that can be imprinted for labeling, decorative or instructional purposes.

Yet another object is to provide an ultrasonically laminated article that is engineered to delaminate after the treatment composition is consumed and the use thereof completed, primarily for the purpose of insuring disposability after use.

These and other objects will become readily apparent from a reading of the detailed description of the invention that follows.

The present invention relates to a flexible, substantially dry, disposable article suitable as a personal care article for cleaning and conditioning skin, and for cleaning, sanitizing and disinfecting hard surfaces, said article comprising:

1. a first water insoluble flexible nonwoven thermoplastic outer layer having an outer and an inner surface,

2. a core layer containing a treatment composition adapted for cleaning, sanitizing or disinfecting hard surfaces or for personal cleaning and conditioning and

3. a second water insoluble flexible nonwoven thermoplastic layer having an outer and inner surface positioned so its inner surface is opposite the inner surface of the first layer, the core layer being ultrasonically bonded to and between the first and second layers so as to form a unified article having pin-dot perforations of less than 0.5 millimeters in diameter formed ultrasonically extending through all three of said layers.

In another aspect of the invention, additional layers of thermoplastic materials such as resin bonded polyester, thermally or spun bonded polypropylene or needle punched polypropylene or polyester may be disposed adjacent to the outer layers or used to form the bottom surface or the top surface, of the cleansing article respectively.

Preferably, the first and second outer layers are composed essentially of thermoplastic nonwovens selected from polyesters, polyolefins, vinyl acetate copolymers, and most preferably resin bonded polyester, thermally or spun bonded polypropylene and needle punched polypropylene. The first and second outer layers or additional layers provided on the first and second layers can also be comprised of a mixture of the thermoplastic fibers with abrasive particles. The abrasive particles are added in order to form a rough or abrasive surface preferably on at least one face of the article. An outer layer can also be embossed or debossed ultrasonically so that it exhibits a pattern of raised and depressed areas or surface aberrations as an alternative to including abrasive polymers. This abrasive or embossed surface also serves to enhance the cleaning or exfoliating effect.

The thermoplastic nonwovens utilized for the additional outer layers can be selected so as to provide a soft texture or surface, and be resin bonded, thermal bonded, spun bonded or needle punched materials.

To form the disposable cleaning article of this invention, a core layer is ultrasonically through-bonded while it is positioned between the outer layers using a charge sufficient to achieve the bonding of the elements and to perforate all of the layers effectively. The perforations of the layers have a diameter of less than 0.5 millimeters and are rendered fluid permeable. The perforations help control and meter water absorption, activation and release of the cleansing or treatment component. If more than three layers are present, the bonding and formation of apertures or perforations is achieved in the same manner and to the same effect.

The term “disposable” as used herein means that the sonically bonded and perforated articles are designed to be used a limited number of times and then discarded, preferably less than about 12 to 24 times and most preferably no more than a single usage event. Traditionally, sonically bonding practices have mandated the achievement of solid bond points to prevent de-lamination under normal repetitive wiper use. Through-bonding (causing perforations) has not been practiced or commercialized for that reason. For the purpose of the instant invention articles, all of which are specifically designed for disposability, through-bonding enhances performance and mandates disposability by facilitating controlled delamination to be engineered into the product's design specifications.

The term “substantially dry”, as used herein, means that the articles exhibit a hand feel that may feel slightly lubricious, but not water-wet.

The disposable cleaning article of the invention must comprise a water insoluble inner core element and two outer water insoluble surface contacting elements with at least one outer surface containing elements on each side of the core element wherein the article contains apertures of less than 0.5 millimeters in diameter extending through all three of the elements or layers. The core element may include a cellulosic (paper) or nonwoven web having absorbent capacity, compressibility, controlled retention and dispensing rates and wet strength for use and physical strength. In its preferred embodiment, the nonwoven core should have an absorbent capacity of up to ten grams per gram of core material. The wet wicking rate of the core should be less than about 25 seconds. The base weight of the core should be from about 15 to about 100 grams per square meter. Materials for the core are also selected from the group of cellulosic, natural and thermoplastic nonwovens, which should be of low density, not exceeding 0.5 grams per cubic centimeter.

The core element may include a coating comprising the active ingredients in the form of a substantially dry composition that does not permeate or impregnate either of the outer layers, and effectively resides between the inner surfaces of the first and second or outer layers.

The first and second and any additional outer layers and the core are bonded together to maintain the integrity of the article and to provide the article in the thermoplastic outer layers with apertures such that the layers become fluid permeable. The apertures encourage the introduction of water or other fluids into the core to activate the treatment composition that has been impregnated, coated, sprayed or otherwise incorporated therein. The application of pressure by the hand facilitates and controls the metered release of the activated composition.

The bonding of the core with the outer layers is executed ultrasonically with a charge sufficient to perforate all of the layers completely.

The cleaning articles of the invention contain in the core layer a treatment composition which has been added onto or impregnated into the core layer and is releasably associated therewith.

The treatment composition for the article to be used for personal cleansing can include, in addition to surfactants and preferably lathering surfactants, emollients, lubricants, protectants, deodorants or medicaments.

The apertures need not be but preferably will be uniform in size and shape and have an average diameter of less that 0.5 mm. In addition to the two layers and core, other insoluble layers may be present disposed adjacent to the outer surface(s) of the two layers. These additional layers can include films, abrasives and other non-fibrous materials. The bonding of the multi layers takes place ultrasonically and is regulated so as to bond the layers and to produce apertures or openings extended into and through all of the layers.

The articles intended for cleaning hard surfaces can have incorporated therein compositions as described in U.S. Pat. No. 6,141,644 the entirety of which is incorporated herein by reference.

The treatment composition for the article to be used for cleaning, sanitizing and disinfecting hard surfaces, can include, in addition to surfactants, an antiseptic, antibacterial, wax, waterproofing, polishing or other agents as are conventionally used in cleaning compositions intended for cleaning and protecting hard surfaces in homes, restaurants, hospitals, nursing homes and the like.

In a preferred embodiment for personal skin care, the treatment composition includes a surfactant and at least one member of the group of emollients, lubricants, conditioning agents, protectants, deodorants and medicaments. When the article is exposed to water at the point of use and pressure applied, as by squeezing, an unstable emulsion and in the case of the use of a foaming surfactant, a lather is formed which releases the components of the cleansing treatment composition onto the skin or hair of the individual.

The outer layers of the article of the invention may have different textures and abrasiveness. Differently textured surfaces can be adapted so as to provide an abrasive side for more intensive cleaning and a softer absorbent side for lighter and more gentle cleaning.

The surfactants employed in the treatment compositions are preferably lathering surfactants, but this is not required and may include anionic, cationic, amphoteric and non-ionic surfactants either separately or in combination and include as anionic the phosphates, taurates, sulfates, sarcosinates, isothionates, etc, for example ammonium lawiyl sulfate, sodium lauroyl sarcosinate, sodium lauroyl lactylate, etc.

Nonionic surfactants which can be used include amine oxides, alkoxylated fatty acid esters, polyhydroxy fatty acid amides, alkyl glycosides and the like exemplified by lauramine oxide, sucrose laurate, sucrose cocoate and the like.

Instances of suitable amphoteric surfactants are the amino alkanoates, alkyl amino acetates, hydroxy, sultaines, betaines, etc., as exemplified by sodium-3-dodecyl-aminopropinate, disodium lauroamphodiactetate and sodium lauroampho acetate.

The substantially dry core formed by impregnation, coating or other conventional methods of application with the treatment composition is then interposed between the two water insoluble outer layers, and the resultant lamination is ultrasonically bonded to form a perforated uniform article.

Preferably the article of the invention comprises three discrete elements that have been ultrasonically through-bonded to form a uniform perforated article. For the purpose of sonic bonding, the outer layers are preferably composed of the same thermoplastic fiber materials.

There may as previously noted be additional layers incorporated in the composite and these will be bonded and perforated as a unit.

The disposable articles of the invention can accommodate from 25 to about 300 percent of treatment composition of the resulting structures' total basis weight without any loss of their excellent hand properties including feel, drape, conformability and flexibility.

The disposable articles of the invention are substantially dry and are intended to be wet with water prior to use by immersion in water, by placing it under a stream of water or by placing it in contact with a wet surface or wet skin. The treatment composition is released in active form by squeezing or rubbing the article prior to or during contact of the article with the surface to be treated or cleaned.

In order to describe the invention more fully and not by way of limitation, the following examples are presented.

1. THREE-PLY HARD SURFACE CLEANING CLOTHS—A single, inner absorbent core element comprising a 28# cellulosic material, with a density of less than 0.1 gram per cubic centimeter, is impregnated with 90 grams per square yard of a highly concentrated hard surface surfactant containing treatment composition. This treated core element (weighing approximately 126 grams per square yard) is then interposed between a 1.3 ounces resin-bonded, polyester material and a 1.0 ounce spun-bonded polyester material. The three elements are then bonded together ultrasonically so as to form a flexible entity having perforated bonding points not exceeding 0.5 mm in diameter arranged in a uniform pin-dot pattern that are visible on the spun-bonded polyester side only. The resulting cloth is then placed under running water and squeezed several times to provide a rich, lathering foam. This process was repeated approximately twelve times, at which point the lathering foam diminished significantly, and the article exhibited obvious signs of de-lamination.

2. FOUR PLY HARD SURFACE CLEANING CLOTHS—A single, inner absorbent core element comprising a 28# cellulosic material, with a density of less than 0.1 grams per cubic centimeter, is impregnated with 90 grams per square yard of a highly concentrated hard surface surfactant containing treatment composition. This treated core element (weighing approximately 126 grams per square yard) is then interposed between a 1.3 ounce resin-bonded polyester material which is interfaced with a 0.5 milligram polyethylene film and the 1.0 ounce spun-bonded polyester material. These four layers are then bonded together ultrasonically so as to form a flexible article having perforated bonding points arranged in a uniform pin-dot pattern through which fluids can pass into the treated core and the activated treatment composition can be released. The perforated polyethylene film layer is intended to present a partial fluid barrier that restricts the flow of water in and the outward release of activated treatment. The treated cloth is then saturated by immersion in water and squeezed several times to produce a rich lathering foam. This process was repeated over 24 times before the foaming action began to dissipate, and the article showed visible signs of de-lamination.

3. FOUR PLY HARD SURFACE CLEANING CLOTHS—A single, inner absorbent core element comprising a 28# cellulosic material, with a density of less than 0.1 gram per cubic centimeter is coated in three-quarter inch wide stripes with 60 grams per square yard of a highly concentrated hard surface surfactant containing composition, separated by one-half inch strips having no coating. The core element, treated in this manner is then interposed between a 1.3 ounce resin-bonded polyester material which has been interfaced with a 0.5 milligram polyethylene film and the 1.0 ounce spun-bonded polyester material. The four layers are then bonded together ultrasonically so as to form a flexible entity having perforated bonding points of less than 0.5 millimeter in diameter arranged in a uniform pin-dot pattern through which fluids can pass into the treated core and the activated treatment can be released outwardly. The perforated polyethylene film layer is intended to present a partial fluid barrier that restricts in use the flow of water in and the subsequent release of activated treatment composition. The treated cloth was then saturated with water and squeezed several times to produce a rich lathering foam. This process was repeated over 24 times before the foaming action began to dissipate, and the article is easily delaminated.

4. THREE-PLY SKIN CLEANSING CLOTHS—A single, inner absorbent core element comprising a 2.5 ounce needle punched polypropylene material, with a density of less than 0.7 gram of per cubic centimeter, is impregnated with 124 grams per square yard of a concentrated skin cleansing and conditioning composition. This treated core element (weighing approximately 195 grams per square yard) is then interposed between a 1.3 ounce resin-bonded, polyester material and a 1.0 ounce spun-bonded polyester material. The three elements are then bonded together ultrasonically so as to form a flexible entity having perforated through-bonding points with a diameter of less than 0.5 millimeter arranged in a uniform pin-dot pattern that are visible on the spun-bonded polyester side only. The treated cloth is then saturated under running water and squeezed several times to provide a rich, lathering foam. This process was repeated approximately 12 times, at which point the lathering foam diminished significantly, and the article exhibited some visible delamination.

5. FOUR PLY SKIN CLEANSING CLOTHS—An single, inner absorbent core element comprising a 2.5 ounce needle punched polypropylene material with a density of less than 0.5 gram per cubic centimeter, that is impregnated with 125 grams per square yard of a conventional highly concentrated skin cleansing and conditioning composition. This treated core element (weighing approximately 195 grams per square yard) is then interposed between a 1.3 ounce resin-bonded polyester material which is interfaced with a 0.5 milligram polyethylene film and a 1.0 ounce spun-bonded polyester material. The four layers are then bonded together ultrasonically so as to form a flexible article having perforated through-bonding points with a diameter of less than 0.5 millimeters arranged in a uniform pin-dot pattern through which fluids can pass into the treated core and the activated treatment can be released. The perforated polyethylene film layer presents a partial fluid barrier that restricts the flow of water in and the release of activated treatment out of the article. The treated cloth is then saturated under water and squeezed several times to produce a rich lathering foam. This process was repeated over 24 times before the foaming action began to dissipate, and the article began to show some visible delamination.

6. THREE-PLY SKIN CLEANSING CLOTHS—An inner absorbent core element comprising a 2.5 ounce needle punched polypropylene material with a density of less than 0.5 gram per cubic centimeter, is coated in three-quarter inch wide stripes with 125 grams per square yard of a highly concentrated skin cleansing and conditioning composition. The core element formed in this manner (weighing approximately 195 grams per square yard) is then interposed between a 1.3 ounce resin-bonded polyester material and a 1.0 ounce spun-bonded polyester material. The three layers are then bonded together ultrasonically so as to form a flexible entity having perforated through-bonding points with a diameter of less than 0.5 millimeters arranged in a uniform pin-dot pattern that are visible on the spun-bonded polyester side only. The treated cloth is then saturated under water and squeezed several times to produce a rich lathering foam. The process is repeated approximately 12 times at which point the lathering foam diminished significantly, and the article exhibited some visible delamination.

7. FIVE-PLY SKIN CLEANSING CLOTHS—A single, inner absorbent core element comprising a 2.5 ounce needle punched polypropylene material with a density of less than 0.5 gram per cubic centimeter, is impregnated with 124 grams per square yard of a highly concentrated skin cleansing and condition composition. This treated core element (weighing approximately 195 grams per square yard) is then interposed between a 1.3 ounce resin-bonded polyester material which is interfaced with a 0.5 milligram polyethylene film and the 1.0 ounce spun-bonded polyester material which is also interfaced with a 0.5 milligram polyethylene film. The five layers are then bonded together ultrasonically so as to form a flexible entity having perforated through-bonding points with a diameter of less than 0.5 millimeters arranged in a uniform pin-dot pattern through which fluids can pass gradually into the treated core and the activated treatment can be controllably released. The perforated polyethylene film layers present a partial fluid barrier that restricts the flow of water in and the release of activated treatment. The five-ply treated cloth is then saturated in a shower and squeezed several times to produce a rich lathering foam. It was then used to wash one section of the body (e.g. one arm) rinsed, squeezed and used to cleanse another portion of the body. This process was repeated over 20 times over a three to five minute period until the entire body was cleansed before the foaming action began to dissipate.

What is claimed is:

1. A disposable flexible article useful for cleaning, sanitizing and disinfecting hard surfaces comprising:
a) a first water insoluble non woven layer having outer and inner surfaces,
b) a water insoluble non woven core layer containing a treatment composition adapted for cleaning hard surfaces, and
c) a second water insoluble non woven layer having outer and inner surfaces wherein the core layer is positioned between the inner surfaces of said first and second layers and the first, second and intermediate core layer are ultrasonically through bonded to form a unified article having perforations of less than 0.5 mm in diameter extending through all of said layers, said perforations having been formed during said ultrasonic bonding.

2. A disposable flexible article according to claim 1 wherein said treatment composition contains surfactant selected from the group consisting of anionic, nonionic, amphoteric surfactants and mixtures thereof.

3. A disposable flexible article according to claim 2 wherein said anionic surfactant is a member selected from the group consisting of sarcosinates, sulfates, isothionates, phosphates, taurates, lactylates, glutamates, and mixtures thereof.

4. A disposable flexible article according to claim 2 wherein said non-ionic surfactant is a member selected from the group consisting of amine oxides, alkyl glucosides, alkyl polyglucosides, polyhydroxy fatty acid amides, alkoxylated fatty acid esters, sucrose esters, and mixtures thereof.

5. A disposable flexible article according to claim 2 wherein said amphoteric surfactant is a member selected from the group consisting of betaines, sultaines, hydroxysultaines, alkyliminoacetates, iminodialkanoates, aminoalkanoates, and mixtures thereof.

6. A disposable flexible article according to claim 1 wherein said article is substantially dry prior to use.

7. A disposable flexible article according to claim 1 wherein said article readily delaminates following use thereof.

8. A disposable flexible article according to claim 1 wherein at least one additional layer is present having been applied to the outer surface of at least one of said first and second layers prior to ultrasonic bonding.

9. A disposable flexible article according to claim 1 wherein at least one of said first and second layers contains an abrasive material.

10. A disposable flexible article according to claim 8 wherein said additional layer has a different texture than said first or second layer.

11. A disposable flexible article according to claim 10 wherein said different texture has been formed by embossing or deembossing.

12. A disposable flexible article according to claim 8 wherein said additional layer serves a partial fluid barrier.

13. A disposable flexible article according to claim 1 wherein said treatment composition is present in an amount of from 25 to 300% of the article's total basis weight.

14. A method of manufacturing a disposable flexible article according to claim 1 comprising the steps of adding a treatment composition onto or impregnating a treatment composition into said core layer, placing the treated core layer between said first and second layers, ultrasonically through bonding said layers to simultaneously form apertures having a diameter of less than 0.5 mm throughout said article.

15. A method of cleaning a hard surface with an article according to claim 1 comprising the steps of wetting said article with water and contacting the surface to be cleaned with said article.
